(19) United States
(12) Patent Application Publication
(10) Pub. No.: US 2022/0259532 A1
(43) Pub. Date: Aug. 18, 2022

(54) METHOD FOR CONTROL OF A BIOPROCESS BY SPECTROMETRY AND TRAINED MODEL AND CONTROLLER THEREFORE

(71) Applicant: Cytiva Sweden AB, Uppsala (SE)

(72) Inventors: Magnus Raberg, Uppsala (SE); Tobias Soderman, Uppsala (SE); Andreas Borgstrom, Uppsala (SE); Helena Ohrvik, Uppsala (SE)

(21) Appl. No.: 17/618,352

(22) PCT Filed: Jun. 23, 2020
(86) PCT No.: PCT/EP2020/067415

§ 371 Date: Dec. 10, 2021

(30) Foreign Application Priority Data
Jun. 25, 2019 (GB) 1909082.8

(51) Int. Cl. C12M 1/36; G05B 17/02

(52) U.S. Cl. CPC C12M 1/36; G05B 17/02

(57) ABSTRACT

The present invention relates to a computer implemented method performed by a controller (C) configured to control a bioprocess comprised in a bioreactor (BR), the method comprising obtaining (410) measurement results by performing spectroscopy of a bioprocessing fluid (FL) comprised in the bioreactor (BR), generating bioprocessing parameters using the measurement results, one or more bioprocessing target parameters and one or more trained models, and, controlling the bioprocess using the generated bioprocessing parameters. The method wherein the one or more trained models are neural networks, wherein the measurement results comprise a spectrum, wherein the spectrum is split to a number N parts used to calculate N average values, wherein the N average values and the corresponding values of bioprocessing parameters are used § as features in the neural network.

### TECHNICAL FIELD

The present invention relates to a method for controlling a bioprocess. The invention further relates to a controller and a system.

### BACKGROUND

The biotechnology industry frequently uses bioreactors for performing a bioprocess such as cultivation of cells. Performing a bioprocess typically involves controlling flow of one or more additive fluids and/or one or more additive gases to a bioprocessing fluid such as a cell culture. An example of an additive fluid may be glucose. An example of an additive gas may be oxygen.

In one example, during a typical bioprocessing manufacturing process, there is typically a need to monitor process properties/bioprocessing variables in the bioprocessing fluid. For example, the process properties/variables that need to be monitored may include glucose concentration, lactose/lactate concentration, viable cell concentration, temperature, fluid and gas pressure, fluid pH, fluid conductivity, and the like.

A problem with controlling bioprocesses is that the nature of the monitored process properties/bioprocessing variables and/or the nature of the bioprocess may prevent the use of sensors in the bioprocessing fluid, e.g. when the bioprocess involves cell cultivation.

In conventional setups, samples of the bioprocessing fluid are periodically taken and analyzed outside of the bioreactor, so called off-line measurements/analysis, to determine process properties/bioprocessing variables in the bioprocessing fluid. This has the drawback of being a complex and work intensive activity requiring an operator present to generate the samples.

Some conventional solutions determine a model for predicting the process properties/bioprocessing variables.

One example is shown in “In Situ Monitoring of CHO Cell Culture Medium Using Near-Infrared Spectroscopy”, Robert A. Mattes, Denise Root, David Chang, Mike Molony, and Mahalia Ong, BioProcess International, January 2007.

A further problem is that a flow of the one or more additive fluids and/or the one or more additive gases to the bioprocessing fluid need to be controlled dependent on the process properties/bioprocessing variables in the bioprocessing fluid.

Some conventional solutions, e.g. as described in the example above, have used evaluation models for spectroscopic data collected from a large reference set of bioprocessing conditions, where the manually determined process properties/bioprocessing variables are correlated with corresponding spectroscopic data from readings obtained from sensors in the bioprocessing fluid. This has the drawback of needing the generation of extensive data sets that models can be based on. A further drawback is that the generated model is commonly, with acceptable accuracy, only applicable to the very bioprocess scale and bioreactor system in which the data set was generated.

There is therefore a need for an improved method for controlling a bioprocess.

### OBJECTS OF THE INVENTION

An objective of embodiments of the present invention is to provide a solution which mitigates or solves the drawbacks and problems described above.

### SUMMARY OF THE INVENTION

The above objective is achieved by the subject matter described herein. Further advantageous implementation forms of the invention are further defined herein.

According to a first aspect of the invention, the above mentioned and other objectives are achieved by a computer implemented method performed by a controller configured to control a bioprocess comprised in a bioreactor, the method comprising obtaining measurement results by performing spectroscopy of a bioprocessing fluid comprised in the bioreactor (BR), generating bioprocessing parameters using the measurement results, one or more bioprocessing target parameters and one or more trained models, and, controlling the bioprocess using the generated bioprocessing parameters.

At least one advantage of the first aspect is that output of the bioprocess can be improved by providing a desired outcome, instead of performing the cumbersome work of manually performing off-line measurements and iterating adjustments of bioprocessing system control parameters.

According to a second aspect of the invention, the above mentioned and other objectives are achieved by a controller, the controller comprising processing circuitry; and a memory, said memory containing instructions executable by said processor, whereby said controller is operative to perform any of the method steps according to the first aspect.

According to a third aspect of the invention, the above mentioned and other objectives are achieved by a bioprocessing system comprising a sensor configured to perform spectroscopy of a bioprocessing fluid and provide measurement results comprised in a control signal, a first controllable flow unit configured to control a flow of one or more additive gases to a bioreactor in response to control signals, a second controllable flow unit configured to control a flow of one or more additive fluids to the bioreactor in response to control signals, the controller according to the second aspect further configured to receive/send control signals to/from the sensor, the first controllable flow unit and the second controllable flow unit.

According to a fourth aspect of the invention, the above mentioned and other objectives are achieved by a computer program comprising computer-executable instructions for causing a controller, when the computer-executable instructions are executed on processing circuitry comprised in the controller, to perform any of the method steps according to the first aspect.

According to a fifth aspect of the invention, the above mentioned and other objectives are achieved by a computer program product comprising a computer-readable storage medium, the computer-readable storage medium having the computer program according to the fourth aspect embodied therein.

The advantages of the second, third, fourth and fifth aspect of the invention are at least the same as for the first aspect.

It is noted that embodiments of the present disclosure relate to all possible combinations of features recited in the claims. The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a bioprocessing system according to one or more embodiments of the present disclosure.

FIG. 2 illustrates functional modules of a controller according to one or more embodiments of the present disclosure.

FIG. 3 shows the controller according to one or more embodiments of the present disclosure.

FIG. 4 shows a flowchart of a method according to one or more embodiments of the present disclosure.

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### DETAILED DESCRIPTION

An “or” in this description and the corresponding claims is to be understood as a mathematical OR which covers “and” and “or”, and is not to be understand as an XOR (exclusive OR). The indefinite article “a” in this disclosure and claims is not limited to “one” and can also be understood as “one or more”, i.e., plural.

In this disclosure, the terms bioprocessing variables denotes properties of a bioprocess obtainable from measurements, e.g. in the form of an absorption or scattering spectrum, as well as bioprocessing system control parameters used to control the bioprocessing system and/or the bioprocess. Examples of bioprocessing variables indicative of properties of a bioprocess may e.g. be any selection of any of glucose concentration, lactate concentration and viable cell density. An example of bioprocessing variables indicative of bioprocessing system control parameters may be gassing or additive gas flow rate, additive fluid flow rate, bioprocessing time, ambient air temperature and temperature of the bioprocessing fluid FL.

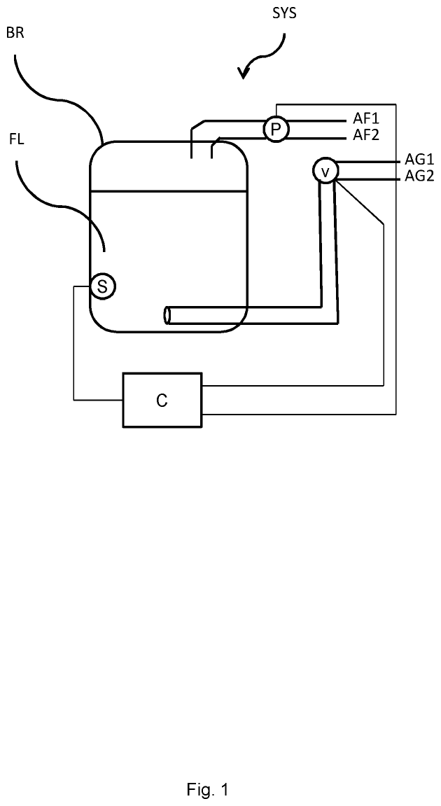

FIG. 1 shows a bioprocessing system SYS according to one or more embodiments of the present disclosure. The bioprocessing system SYS comprises a sensor S configured to perform spectroscopy of a bioprocessing fluid FL and provide measurement results comprised in a control signal. The sensor S may e.g. be a probe designed for to be in direct contact with the bioprocess fluid and/or configured to generate an absorbance spectrum, e.g. in the near infrared, NIR, wave length region. Alternatively or additionally the probe may e.g. be a probe designed to be in direct contact with the bioprocess fluid and/or configured to generate a Raman spectrum, e.g. a spectroscopy sensor, as commercially available from Hellma.

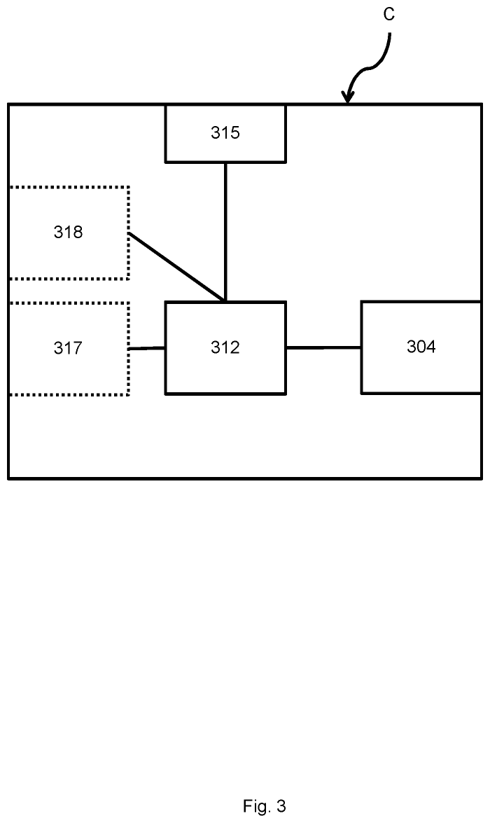

The bioprocessing system SYS further comprises a controller C, further described in relation to FIG. 3. The bioprocessing system SYS further comprises a first controllable flow unit V configured to control a flow of one or more additive gases AG1-AG2 to a bioreactor BR in response to received control signals. The first controllable flow unit V may e.g. comprise one or more electrically controlled valve units configured to control the flow of one or more additive gases AG1-AG2 to a bioreactor BR by, at least partially, opening/closing one or more valves in response to the control signals. The gases may e.g. be oxygen O₂ and/or carbon dioxide CO₂. The bioprocessing system SYS further comprises a second controllable flow unit P configured to control a flow of one or more additive fluids AF1-AF2 to a bioreactor BR in response to control signals. The second controllable flow unit P may e.g. comprise one or more pumps and/or one or more valve units. The one or more additive fluids AF1-AF2 may e.g. comprise any one of glucose, lactose/lactate, amino acids, carbohydrates, vitamins, minerals, growth factors or hormones.

The controller C is communicatively coupled to the sensor S, the first controllable flow unit V and the second controllable flow unit P. The controller is further configured to receive/send control signals to/from the sensor S, the first controllable flow unit V and the second controllable flow unit P.

The bioprocessing system SYS may further optionally be coupled to a bioreactor BR, as shown in FIG. 1. The first controllable flow unit V and the second controllable flow unit P may be couplable to one or more inlets of the bioreactor BR, thereby allowing the one or more additive gases AG1-AG2 and/or the one or more additive fluids AF1-AF2 to mix into the bioprocessing fluid FL. The sensor S may be configured to be comprised in the bioreactor BR or configured to be inserted into the bioreactor BR such that the sensor S is at least in part in contact with the bioprocessing fluid FL.

In one example, the controller C comprises one or more trained models, used to generate and/or predict bioprocessing parameters, e.g. bioprocessing variables and/or bioprocessing system control parameters. The controller C obtains/receives measurement results in a control signal from the sensor S, e.g. a NIR spectrum/spectra. The measurement results are obtained by the sensor S by performing spectroscopy of the bioprocessing fluid FL comprised in the bioreactor BR. The controller C then inputs the measurement results into the one or more trained models together with one or more bioprocessing target parameters generate/predict bioprocessing parameters.

The generated bioprocessing parameters may be indicative of bioprocessing variables describing properties of the bioprocess and/or bioprocessing fluid FL. In one example, the generated bioprocessing variables are indicative of a selection of any of glucose concentration, lactose concentration, glutamine concentration, glutamate concentration, osmolality, desired product concentration (such as. Immunoglobulin or IgG), and viable cell density.

The generated bioprocessing parameters may further be indicative of bioprocessing system control parameters that may e.g. define how much the flow of the one or more additive gases AG1-AG2 or the flow of the one or more additive fluids AF1-AF2 should be adapted in response to the measurement results/spectrum. The controller C may further be configured to control the bioprocess using the generated bioprocessing parameters.

In one example, the generated bioprocessing system control parameters comprises proportional-integral-derivative, PI D, controller parameters configured to control the operation of a PID regulator comprised in the controller C or arranged separately to the controller C. The PID regulator may further be communicatively coupled to a selection of any of the controller C, the first controllable flow unit V and the second controllable flow unit P to control flow of the one or more additive gases AG1-AG2 or the flow of the one or more additive fluids AF1-AF2 to the bioreactor BR. Any other type of suitable controller and controller parameters can be envisioned without departing from the present disclosure.

In a further example, the one or more trained models are generated by training a neural network. A reference data set may be obtained by manually determining process properties/bioprocessing variables and/or bioprocessing system control parameters for a particular bioprocess of a particular scale in a particular bioreactor of a particular size. The reference data set may further be expanded. Expanding the reference data set may e.g. be performed by increasing or decreasing the concentration or concentration of various components in the bioprocessing fluid FL, e.g. by spiking the bioprocessing fluid FL or diluting the bioprocessing fluid FL and obtain measurement results of light reflection or absorption. This way, spectral measurement data over a range spanning outside normal biological range used in upstream cultivation can then be generated.

Expanding the reference data set may optionally further be performed by systematically varying the unwanted bioprocessing variables in a systematic manner, e.g. by applying design of experience approach where variables such as fluid and gas concentrations, pH or temperature are systematically varied and spectroscopic data for the different variations are obtained.

Expanding the reference data set may further be performed by repeating the above mentioned steps for bioreactors having different volumes. The reference data set resulting from expanding the starting data set using any of the method above may further be analyzed using orthogonal partial least squares, O-PLS, analysis to filter away effects due to the bioreactor reactor scale/volume.

In other words, one or more trained models may be generated that receives input variables and/or measurement results and generates/predicts bioprocessing parameters. The model is trained/generated by providing the reference data as input variables and then adapting the model such that the output substantially matches reference bioprocessing parameters/reference output of the reference data set.

In one example, the one or more trained models predict that glucose concentration is decreasing and is not maintained at a constant level by the current bioprocessing system control parameters. An updated set of bioprocessing system control parameters may then be generated by determining that the reduced glucose concentration predicted by the model indicates that a log phase has been entered by the bioprocess, and that increased proportional terms, e.g. of a proportional-integral-derivative PID controller, needs to be generated for the updated set of bioprocessing system control parameters.

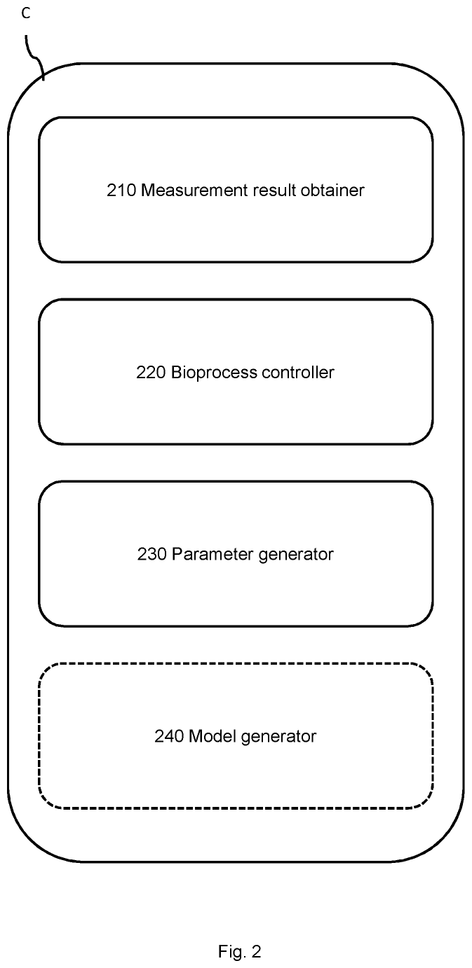

FIG. 2 illustrates functional modules of the controller C according to one or more embodiments of the present disclosure. It is appreciated that the functionality of the controller C may be distributed over fewer or further functional modules depending on the application, and that the purpose of the concept of functional modules is used for illustrative purposes. In other words, the functionality of the controller may be concentrated to a single functional module or distributed over a plurality of functional modules without departing from the scope of the present disclosure.

In one embodiment, the controller C comprises a measurement result obtainer module 210. The measurement result obtainer module 210 is primarily configured to obtain measurement results by performing spectroscopy of the bioprocessing fluid FL. The measurement results are typically obtained by receiving a control signal from the sensor S. The control signal typically comprises an indication of the measurement results resulting from performing spectroscopy of the bioprocessing fluid FL, e.g. indicative of quantitative measurements of the reflection or transmission properties of the bioprocessing fluid FL as a function of wavelength of emitted light by the sensor S.

In one example, the measurement result comprises a generated spectrum of the bioprocess fluid, the spectrum showing the specific reflection/absorbance values of spectrum of light, e.g. a Near Infra-Red, NIR. In other words, reflection/absorbance values as a function of the wavelength of the emitted light. The reflection/absorbance values at specific wavelengths can be related to the molecular structures present in the bioprocess fluid and is accordingly indicative of the chemical composition of the fluid. The spectrum resulting from a full NIR scan may include a wavelength range 0.75-1.4 μm. If one of the bioprocessing variables includes glucose concentration, the spectrum resulting from a NIR scan may preferably include a wavelength range between 5450-4497 cm⁻¹ and/or between 7501-5630 cm⁻¹. If one of the bioprocessing variables includes Lactate concentration, the spectrum resulting from a NIR scan may preferably include a wavelength range between 8921-7146 cm⁻¹.

In one embodiment, the controller C further comprises a bioprocess controller module 220. The bioprocess controller module 220 is typically configured to control a flow of one or more additive fluids AF1-AF2 and/or to control a flow of one or more additive gases AG1-AG2 to the bioreactor BR and/or the bioprocessing fluid FL of the bioreactor BR. The flow is typically controlled in response to values of bioprocessing variables generated or predicted by the one or more trained models.

In one example, the controller C controls the flow of one or more additive gases AG1-AG2 to the bioreactor BR by sending a control signal to the first controllable flow unit V. The first controllable flow unit V typically comprises a valve unit and the control signal activates or controls one or more valves of the valve unit. E.g. the flow of oxygen into the bioprocessing fluid FL is controlled to a certain volume per time unit.

In one example, the controller C controls the flow of one or more additive fluids AF1-AF2 to the bioreactor BR by sending a control signal to the second controllable flow unit P. The second controllable flow unit P typically comprises a pump and the flow of the pump is controlled by the control signal. E.g. the flow of glucose into the bioprocessing fluid FL is controlled to a certain volume per time unit.

In one example, the controller C controls the flow of one or more additive gases AG1-AG2 and/or one or more additive fluids AF1-AF2 to the bioreactor BR by sending bioprocessing system control parameters, e.g. in the form of proportional-integral-derivative controller parameters to one or more PID controllers.

In one embodiment, the controller C comprises a bioprocessing parameter generator module 230. The bioprocessing parameter generator module 230 is typically configured to generate bioprocessing parameters based on the measurement results of the sensor S and/or one or more bioprocessing target parameters and/or generated/predicted bioprocessing variables of the one model. The bioprocessing system control parameters typically reflect the responsiveness, of the bioprocess controller module 220 and/or one or more controllers external to the controller C, which are controlling the flow of one or more additive gases AG1-AG2 and/or one or more additive fluids AF1-AF2 to the bioreactor BR, in response to changes in measurement result values and/or generated/predicted bioprocessing variables, e.g. a decreased concentration of glucose in the bioprocessing fluid FL or density of viable cells in the bioprocessing fluid FL.

In one example, a change in reflection or transmission properties of the bioprocessing fluid FL indicated by the measurement results may indicate that the bioprocess is in a particular phase of the bioprocess, when the concentration of glucose in the bioprocessing fluid FL may drop rapidly, e.g. a log phase. The parameter generator module 230 may then generate bioprocessing system control parameters providing a higher responsiveness to concentration of glucose. E.g. parameters indicating increased proportional terms of a PID controller.

In one example, a change in reflection or transmission properties of the bioprocessing fluid FL indicated by the measurement results may indicate that the bioprocess is in a particular phase of the bioprocess, when the concentration of glucose in the bioprocessing fluid FL is constantly offset, e.g. due to a residual error. The parameter generator module 230 may then generate control parameters providing a higher responsiveness to residual error. E.g. control parameters indicating increased integral terms of a PID controller.

In one example, a change in reflection and/or transmission properties of the bioprocessing fluid FL indicated by the measurement results may indicate that the bioprocess is in a particular phase of the bioprocess, when the concentration of glucose in the bioprocessing fluid FL is constantly offset, e.g. due to a residual error. The parameter generator module 230 may then generate bioprocessing system control parameters providing a higher responsiveness to a rate of error change. E.g. parameters indicating increased derivative terms of a PID controller.

In one embodiment, the controller C further comprises an optional model generator module 240. The model generator module 240 may generate the one or more trained models by training one or more nominal models using the reference data set to, as further described in relation to FIG. 1. The one or more trained models are trained/generated by providing the reference data as input variables and then adapting the model such that the output substantially matches reference bioprocessing parameters/reference output of the reference data set.

A reference data set may be generated by proven methods in order to determine the reference value for various bioprocessing variables/parameters at various time points of the bioprocess. The proven methods may involve determining measurement results by measuring reflection and/or transmission properties of the bioprocessing fluid FL as reference measurement results and simultaneously determine values of bioprocessing variables, typically via manual “off-line” measurements and/or recording the used bioprocessing system control parameters, such as responsiveness etc.

In one example, this involves to determine reference data by determining a glucose concentration of the bioprocessing fluid FL together with the currently used bioprocessing system control parameters. An example of conventional methods performing multivariate data analysis, MVDA, employing unsupervised principal component analysis, PCA, and partial least squares regression methods, PLS, for prediction of multiple cultivation variables during bioprocess-monitoring can be found in “Chemometrics and in-line near infrared spectroscopic monitoring of a biopharmaceutical Chinese hamster ovary cell culture: prediction of multiple cultivation variables”, Clavaud M, Roggo Y, Von Daeniken R, Liebler A, Schwabe J O, Talanta 26 Mar. 2013, 111:28-38.

The reference data set can then be transformed into one or more trained computer models by means of analyzing the absorbance as a function of wavelength and corresponding determined bioprocessing parameter values, e.g. indicative of process time point, and relating this absorbance to the parameter values determined by the proven methods.

FIG. 3 shows the controller C according to one or more embodiments of the present disclosure. The controller C may be in the form of e.g. an Electronic Control unit, a server, an on-board control unit, a stationary computing device, a laptop control unit, a tablet control unit, a handheld control unit, a wrist-worn control unit, a smart watch, a smartphone or a smart TV. The controller C may comprise processing circuitry 312 communicatively coupled to a communications interface, e.g. a transceiver 304, configured for wired or wireless communication. The controller C may further comprise at least one optional antenna (not shown in figure). The antenna may be coupled to the transceiver 304 and is configured to transmit and/or emit and/or receive wired or wireless signals in a communication network, such as WiFi, Bluetooth, 3G, 4G, and 5G etc. In one example, the processing circuitry 312 may be any of a selection of a processor and/or a central processing unit and/or processor modules and/or multiple processors configured to cooperate with each-other. Further, the controller C may further comprise a memory 315 communicatively coupled to the processing circuitry 312. The memory 315 may e.g. comprise a selection of a hard RAM, disk drive, a floppy disk drive, a flash drive or other removable or fixed media drive or any other suitable memory known in the art. The memory 315 may contain instructions executable by the processing circuitry to perform any of the steps or methods described herein. The processing circuitry 312 may be communicatively coupled to a selection of any of the transceiver 304 and the memory 315. The controller C may be configured to send/receive control signals directly to/from any of the above mentioned units or to external nodes or to send/receive control signals via a wired and/or wireless communications network.

The wired/wireless transceiver 304 and/or a wired/wireless communications interface may be configured to send and/or receive data values or parameters as a signal to or from the processing circuitry 312 to or from other external nodes.

In an embodiment, the transceiver 304 communicates directly to external nodes/units or via the wireless communications network. In one example, control parameters are sent to an external PID controller.

In one or more embodiments the controller C may further comprise an input device 317, configured to receive input or indications from a user and send a user input signal indicative of the user input or indications to the processing circuitry 312.

In one or more embodiments the controller C may further comprise a display 318 configured to receive a display signal indicative of rendered objects, such as text or graphical user input objects, from the processing circuitry 312 and to display the received signal as objects, such as text or graphical user input objects.

In one embodiment the display 318 is integrated with the user input device 317 and is configured to receive a display signal indicative of rendered objects, such as text or graphical user input objects, from the processing circuitry 312 and to display the received signal as objects, such as text or graphical user input objects, and/or configured to receive input or indications from a user and send a user-input signal indicative of the user input or indications to the processing circuitry 312.

In a further embodiment, the controller C may further comprise and/or be coupled to one or more additional sensors (not shown in the figure) configured to receive and/or obtain and/or measure physical properties pertaining to the bioprocessing system SYS and send one or more sensor signals indicative of the physical properties to the processing circuitry 312. An example of such an additional sensor may be an ambient air pressure sensor configured to measure the ambient air pressure where the bioprocessing system SYS is located. A further example of such an additional sensor may be an ambient air temperature sensor configured to measure the ambient air pressure where the bioprocessing system SYS is located.

In one or more embodiments, the processing circuitry 312 is further communicatively coupled to the memory 315, the transceiver 304, the input device 317 and/or the display 318 and/or the additional sensors and/or the sensor S.

In embodiments, the communications network communicate using wired or wireless communication techniques that may include at least one of a Local Area Network (LAN), Metropolitan Area Network (MAN), Global System for Mobile Network (GSM), Enhanced Data GSM Environment (EDGE), Universal Mobile Telecommunications System, Long term evolution, High Speed Downlink Packet Access (HSDPA), Wideband Code Division Multiple Access (W-CDMA), Code Division Multiple Access (CDMA), Time Division Multiple Access (TDMA), Bluetooth®, Zigbee®, Wi-Fi, Voice over Internet Protocol (VoIP), LTE Advanced, IEEE802.16m, WirelessMAN-Advanced, Evolved High-Speed Packet Access (HSPA+), 3GPP Long Term Evolution (LTE), Mobile WiMAX (IEEE 802.16e), Ultra Mobile Broadband (UMB) (formerly Evolution-Data Optimized (EV-DO) Rev. C), Fast Low-latency Access with Seamless Handoff Orthogonal Frequency Division Multiplexing (Flash-OFDM), High Capacity Spatial Division Multiple Access (iBurst®) and Mobile Broadband Wireless Access (MBWA) (IEEE 802.20) systems, High Performance Radio Metropolitan Area Network (HIPERMAN), Beam-Division Multiple Access (BDMA), World Interoperability for Microwave Access (Wi-MAX) and ultrasonic communication, etc., but is not limited thereto.

Moreover, it is realized by the skilled person that the control unit CU may comprise the necessary communication capabilities in the form of e.g., functions, means, units, elements, etc., for performing the present solution. Examples of other such means, units, elements and functions are: processors, memory, buffers, control logic, encoders, decoders, mapping units, multipliers, decision units, selecting units, switches, modulators, demodulators, inputs, outputs, antennas, amplifiers, receiver units, transmitter units, DSPs, power supply units, power feeders, communication interfaces, communication protocols, etc. which are suitably arranged together for performing the present solution.

Especially, the processing circuitry of the present disclosure may comprise one or more instances of a processor, processor modules and multiple processors configured to cooperate with each-other, Central Processing Unit (CPU), a processing unit, a processing circuit, a processor, an Application Specific Integrated Circuit (ASIC), a microprocessor, a Field-Programmable Gate Array (FPGA) or other processing logic that may interpret and execute instructions. The expression “processing circuitry” and/or “processing means” may thus represent a processing circuitry comprising a plurality of processing circuits, such as, e.g., any, some or all of the ones mentioned above. The processing means may further perform data processing functions for inputting, outputting, and processing of data comprising data buffering and device control functions, such as call processing control, user interface control, or the like.

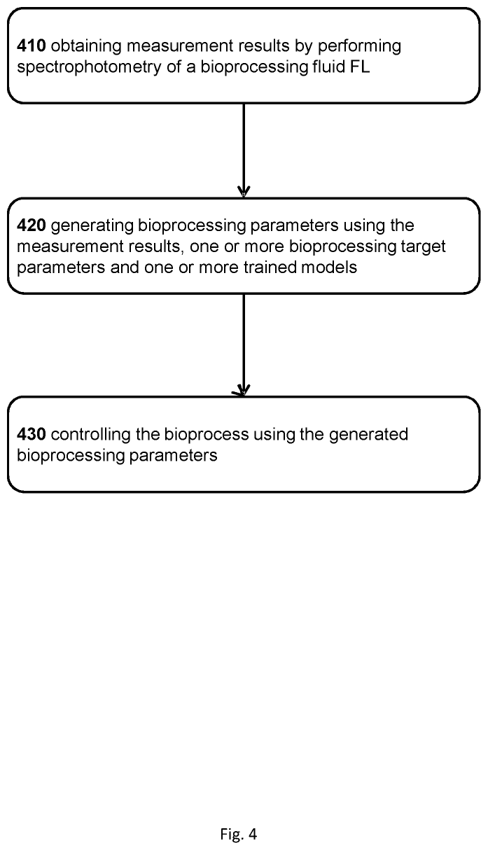

FIG. 4 shows a flowchart of a method 400 according to one or more embodiments of the present disclosure. A computer implemented method 400 is provided and performed by a controller C configured to control a bioprocess, e.g. comprised in a bioreactor BR. The method comprises:

Step 410: obtaining measurement results by performing spectroscopy of a bioprocessing fluid FL. The measurement results are typically obtained by receiving a control signal from the sensor S. The control signal being indicative of the measurement results, e.g. reflection or transmission properties of the bioprocessing fluid FL as a function of wavelength, as further described in relation to FIG. 2.

In one example, the bioprocess is a cell cultivation process and the measurement results are indicative of transmission properties of the bioprocessing fluid FL as a function of wavelength, e.g. intensity of near infrared light, NIR, as a function of wavelength. The wavelengths of the NIR light may typically be comprised in a range of 780 nm to 2500 nm or 0.75-1.4 μm.

Step 420: generating bioprocessing parameters using the measurement results, one or more bioprocessing target parameters and one or more trained models.

Additionally or alternatively, the generated bioprocessing parameters comprise bioprocessing variables and/or bioprocessing system control parameters indicative of a selection of any of glucose concentration, lactose concentration, ammonia concentration, glutamine concentration, glutamate concentration, product concentration and viable cell density of the bioprocessing fluid FL; one or more target flow of one or more additive gases AG1-AG2; one or more target flow of one or more additive fluids AF1-AF2 and bioprocessing system control parameters.

The bioprocessing system control parameters typically reflect the responsiveness to changes in measurement result values, of the bioprocess controller module 220 and/or one or more controllers external to the controller C controlling the flow of one or more additive gases AG1-AG2 and/or one or more additive fluids AF1-AF2 to the bioreactor BR. E.g. responsiveness to a decreased concentration of glucose in the bioprocessing fluid FL or density of viable cells in the bioprocessing fluid FL. Further details and examples of generating control parameters are further described in relation to FIG. 2.

Alternatively or additionally, the generated bioprocessing system control parameters comprise controller parameters. E.g. a selection of any of proportional, integral, and derivative terms of a proportional-integral-derivative, PID, controller.

Additionally or alternatively, the bioprocessing target parameters comprise target values of a selection of any of desired product concentration and viable cell density.

Additionally or alternatively, the one or more trained models are generated by training machine learning models for each of the bioprocessing target parameters using a training data set, wherein the training data set comprises measurement results obtained by performing NIR spectroscopy of the bioprocessing fluid (FL) associated with corresponding values of bioprocessing parameters.

Additionally or alternatively, the one or more trained models are trained on data obtained using smaller scale, such as lab scale, bioreactors and applied on larger scale bioreactors, such as production scale, with kept quality, such as R-squared. In one example, the scale between the smaller scale bioreactor and the production scale bioreactor, is in the order of 2 to 12 times. E.g. training the model using data obtained using a lab scale bioreactor having a volume of 0.55 liters and use the trained model to generate bioprocessing parameters for a production scale bioreactor of seven (7) liters.

Additionally or alternatively, the one or more trained models are neural networks, wherein the measurement results comprise a spectrum, wherein the spectrum is split to a number N parts used to calculate N average values, wherein the N average values and the corresponding values of bioprocessing parameters are used as features in the neural network.

In one example a trained model is provided for generation/prediction of each of the bioprocessing variables glucose concentration, lactose concentration, glutamine concentration, glutamate concentration, Osmolality, desired product concentration (such as. Immunoglobulin or IgG), and viable cell density. The measurement result of a spectrum is obtained. A specific part of the spectrum may be used. The spectrum may be normalized using Standard Normal Variate techniques, SNV. The spectrum is then split or segmented into 27 parts, e.g. of equal spectral width. Each part may then be averaged to generate 27 average values. Each of these 27 values may then be fed as features into a Gaussian normalized 1 hidden layer multilayer perceptron, MLP, neural network, e.g. having 15 nodes. After approximately 200 iterations using reference data, the model is trained and capable of generating/predicting bioprocessing variables glucose concentration, lactose concentration, glutamine concentration, glutamate concentration, Osmolality, desired product concentration (such as. Immunoglobulin or IgG), and viable cell density.

Additionally or alternatively, the bioprocessing parameters are generated further using alarm information of a bioprocessing system.

In one example, an alarm indicative of that a hose of one of the one or more additive gases AG1-AG2 has been disconnected. A different set of trained models may then be used, that have been trained to optimize survival time of cells in the bioprocessing fluid FL. In other words, the alarm signal trigger a change to a different set of trained models that aims at optimizing cell survival time rather than desired product concentration and viable cell density.

Step 430: controlling the bioprocess using the generated bioprocessing parameters. Controlling the bioprocess may typically comprise controlling the flow of one or more additive gases AG1-AG2 and/or one or more additive fluids AF1-AF2 to the bioreactor BR. Controlling the bioprocess is further described in relation to FIG. 2.

An example of an execution of the above described method can be found in relation to FIG. 1. Further details and examples of the method can, as mentioned, be found in relation to FIG. 2 detailing the functionality of the controller C.

Additionally or alternatively, the bioprocess is further controlled using bioprocessing system characteristics. E.g. to look at the total amount of energy used to run the bioreactor, and then use fluctuations in that energy to predict parameters. The bioprocess may be further controlled using further bioprocessing system characteristics such as agitation, gas flow, pump flow, energy consumption, etc. that can be used to predict a process outcome and a process stage.

In one example, the volume of the bioreactor BR may be used as input to generate further improved bioprocessing parameters

Additionally or alternatively, the bioprocess comprises cell cultivation.

Additionally or alternatively, the one or more bioprocessing target parameters are indicative of a desired product concentration and/or a desired viable cell density and the generated bioprocessing parameters comprises bioprocessing system control parameters to obtain the desired product concentration and/or viable cell density when controlling the bioprocess.

Additionally or alternatively, controlling the bioprocess comprises controlling a flow of one or more additive fluids AF1-AF2. Controlling the flow may comprise controlling the flow of a pump providing additive fluids to the bioreactor BR, as further described in relation to FIG. 2.

Additionally or alternatively, controlling the bioprocess comprises controlling a flow of one or more additive gases AG1-AG2. Controlling the flow may comprise controlling the flow of a pump providing additive fluids to the bioreactor BR, as further described in relation to FIG. 2.

In one embodiment, a controller C is provided. The controller comprises processing circuitry 312; and a memory 315, said memory containing instructions executable by said processor 312, whereby said controller is operative to perform any of the method steps described herein.

In one embodiment, a bioprocessing system SYS is provided. The bioprocessing system SYS comprises a sensor S configured to perform spectroscopy of a bioprocessing fluid FL and provide measurement results comprised in a control signal, a first controllable flow unit V configured to control a flow of one or more additive gases AG1-AG2 to a bioreactor BR in response to control signals, a second controllable flow unit P configured to control a flow of one or more additive fluids AF1-AF2 to a bioreactor BR in response to control signals. The controller C may further be configured to receive/send control signals to/from the sensor S, to/from the first controllable flow unit and to/from the second controllable flow unit (P).

In one embodiment, a computer program is provided and comprises computer-executable instructions for causing the controller C, when the computer-executable instructions are executed on processing circuitry 312 comprised in the controller C, to perform any of the method steps described herein.

In one embodiment, a computer program product is provided and is comprising a computer-readable storage medium. The computer-readable storage medium having the computer program above embodied therein.

Finally, it should be understood that the invention is not limited to the embodiments described above, but also relates to and incorporates all embodiments within the scope of the appended independent claims.

1. A computer implemented method performed by a controller configured to control a bioprocess comprised in a bioreactor, the method comprising:
obtaining measurement results by performing spectroscopy of a bioprocessing fluid comprised in the bioreactor,
generating bioprocessing parameters using the measurement results, one or more bioprocessing target parameters and one or more trained models, and,
controlling the bioprocess using the generated bioprocessing parameters.

2. The method according to claim 1, wherein the generated bioprocessing parameters comprise bioprocessing variables and/or bioprocessing system control parameters indicative of a selection of any of glucose concentration, lactose concentration, ammonia concentration, glutamine concentration, glutamate concentration, product concentration and viable cell density of the bioprocessing fluid; one or more target flow of one or more additive gases; one or more target flow of one or more additive fluids and controller parameters.

3. The method according to claim 1, wherein the bioprocessing target parameters comprise target values of a selection of any of product concentration and viable cell density.

4. The method according to claim 1, wherein the one or more trained models are generated by training machine learning models for each of the bioprocessing target parameters using a training data set, wherein the training data set comprises measurement results obtained by performing NIR spectroscopy of the bioprocessing fluid associated with corresponding values of bioprocessing parameters.

5. The method according to claim 4, wherein the one or more trained models are neural networks, wherein the measurement results comprise a spectrum, wherein the spectrum is split to a number N parts used to calculate N average values, wherein the N average values and the corresponding values of bioprocessing parameters are used as features in the neural network.

6. The method according to claim 1, wherein the bioprocessing parameters are generated further using alarm information of a bioprocessing system.

7. The method according to claim 1, wherein the bioprocess is further controlled using bioprocessing system characteristics.

8. The method according to claim 1, wherein the bioprocess comprises cell cultivation.

9. The method according to claim 1, wherein:
the one or more bioprocessing target parameters are indicative of a desired product concentration and/or a desired viable cell density and the generated bioprocessing parameters comprises bioprocessing system control parameters to obtain the desired product concentration and/or viable cell density when controlling the bioprocess.

10. The method according to claim 1, wherein controlling the bioprocess comprises controlling a flow of one or more additive fluids.

11. The method according to claim 1, wherein controlling the bioprocess comprises controlling a flow of one or more additive gases.

12. The method according to claim 1, wherein the one or more trained models are trained on data obtained using smaller scale bioreactors and applied on larger scale bioreactors.

13. The method according to claim 12, wherein the larger scale bioreactors have a volume 2 to 12 times the volume of the smaller scale bioreactors.

14. A controller, the controller comprising:
processing circuitry; and
a memory, said memory containing instructions executable by said processor, whereby said controller is operative to perform the method steps according claim 1.

15. A bioprocessing systems comprising:
a sensor configured to perform near infrared, NIR, spectroscopy of a bioprocessing fluid and provide measurement results comprised in a control signal,
a first controllable flow unit configured to control a flow of one or more additive gases to a bioreactor in response to control signals,
a second controllable flow unit configured to control a flow of one or more additive fluids to a bioreactor in response to control signals,
the controller according to claim 14 further configured to receive/send control signals to/from the sensor, the first controllable flow unit and the second controllable flow unit.

16. A computer program comprising computer-executable instructions for causing a controller, when the computer-executable instructions are executed on processing circuitry comprised in the controller, to perform any of the method steps according claim 1.

17. A computer program product comprising a computer-readable storage medium, the computer-readable storage medium having the computer program according to claim 16 embodied therein.
